(19) United States
(12) Patent Application Publication
(10) Pub. No.: US 2014/0039584 A1
(43) Pub. Date: Feb. 6, 2014

(54) PORTABLE SELF CONTAINED CHEMICALLY ACTIVATED COOLING THERAPY BLANKET

(71) Applicants: Charles F. Potter, Mequon, WI (US); John T. Ford, Gilford, NH (US)

(72) Inventors: Charles F. Potter, Mequon, WI (US); John T. Ford, Gilford, NH (US)

(73) Assignee: MEDICAL THERMODYNAMICS LLC, Mequon, WI (US)

(21) Appl. No.: 13/567,907

(22) Filed: Aug. 6, 2012

(51) Int. Cl. A61F 7/10

(52) U.S. Cl. 607/114

(57) ABSTRACT

A cooling device includes a tube having an interior formed by a first and second film welded together. The tube has a peelable heat seal connecting a portion of the first film with a portion of the second film. The peelable heat seal divides the interior into a first chamber and a second chamber separated from the first chamber. The interior of the first chamber includes one or more materials which have an endothermic heat of solution in water. The interior of the second chamber contains water. The peelable heat seal has an adhesive force that is overcome by an application of an external force to one of the chambers allowing the material in the first chamber and the water in the second chamber to mix.

### BACKGROUND

Thermal regulation blankets may be used to regulate the temperature of a patient for medical treatment. Medical research has established that the damage caused by traumatic medical events such as heart attacks and strokes, exposure to hostile conditions resulting in sun stroke or heat exhaustion and serious injury from severe wounds or other serious bodily insults can be ameliorated by cooling therapy applied very shortly after the event. Cooling packs are used to cool a patient for comfort or medical related issues. Cooling packs include a chemical mixture that provides an endothermic reaction to create or provide cooling.

### SUMMARY

One embodiment includes a cooling device comprising a tube having an interior formed by a first and second film welded together. The tube has a peelable heat seal connecting a portion of the first film with a portion of the second film. The peelable heat seal divides the interior into a first chamber and a second chamber separated from the first chamber. The interior of the first chamber includes one or more materials which have an endothermic heat of solution in water. The interior of the second chamber contains water. The peelable heat seal has an adhesive force that is overcome by an application of an external force to one of the chambers allowing the material in the first chamber and the water in the second chamber to mix.

Another embodiment includes a chemically activated thermal blanket that includes a rupture resistant polymeric film containing a plurality of sealed pockets which contain cooling tubes. Each tube includes an exterior wall of one or more thermoplastic polymeric films. Each film having a very low moisture transmission rate and a heat sealable interior surface. Each tube is divided into two chambers by a peelable heat seal formed by heat sealing two facing portions of the interior surface or surfaces to each other. One of the chambers contains one or more materials which have an endothermic heat of solution in water and the other of the chambers contains water. The pockets are sized such that pressure can be applied to the contained cooling tubes to cause the peelable heat seal to open and allow the material or materials and water to mix but also sized to resist the fracture of the cooling tubes.

One embodiment includes a method for the manufacture of portable self contained cooling thermal blankets. It involves partially welding the peripheral edges of two pieces of a thermoplastic film with a very low moisture transmission rate and a readily heat sealable surface to form a cooling tube and dividing the cooling tube into two chambers with a peelable heat seal between a portion of the heat sealable surfaces of these pieces, followed by filling one chamber with water and the other with one or more materials which have an endothermic heat of solution in water and sealing these two chambers by completing the peripheral welds. This is followed by placing cooling tubes in the pockets of the planar material described above and sealing the pockets such that it would prevent leakage to the environment of the water of the contained in the cooling tubes should one or more of the cooling tubes rupture. Preferably this planar material is heat sealable and the pockets are heat sealed.

A further embodiment includes the application of cooling therapy to a warm blooded animal through the use of the portable self contained thermal blanket described above. In this procedure sufficient pressure is applied to one or more of the cooling tubes in their respective pockets to cause the peelable heat seal to peel apart allowing the contents of the two chambers to mix with a resultant absorption of heat. The blanket is placed in contact with this warm blooded animal. In a preferred embodiment the blanket is placed in contact with a human. In another aspect of one embodiment this blanket is placed in contact with the torso of this warm blooded animal such that its core temperature is lowered.

In a further aspect of one embodiment, one or more of the pockets of the thermal blanket are equipped with temperature sensors and these sensors are monitored to determine if and when the peelable heat seals in additional cooling tubes should be caused to peel.

### BRIEF DESCRIPTION OF THE DRAWINGS

FIG. 1 is a plan view of a cooling tube.

FIG. 2 is a plan view of the cooling tube of FIG. 1 in an opened configuration.

FIG. 3 is a plan view of a thermal blanket.

FIG. 4 is a plan view of a thermal blanket behind a patient.

FIG. 5 is a plan view of a thermal blanket wrapped about a patient.

FIG. 6 is a cross section taken generally along line 6-6 of FIG. 3 with the cooing tube heat seal closed.

FIG. 7 is a cross section along line 7-7 of FIG. 3 of the thermal blanket with the cooling tube heat seal opened.

### DETAILED DESCRIPTION

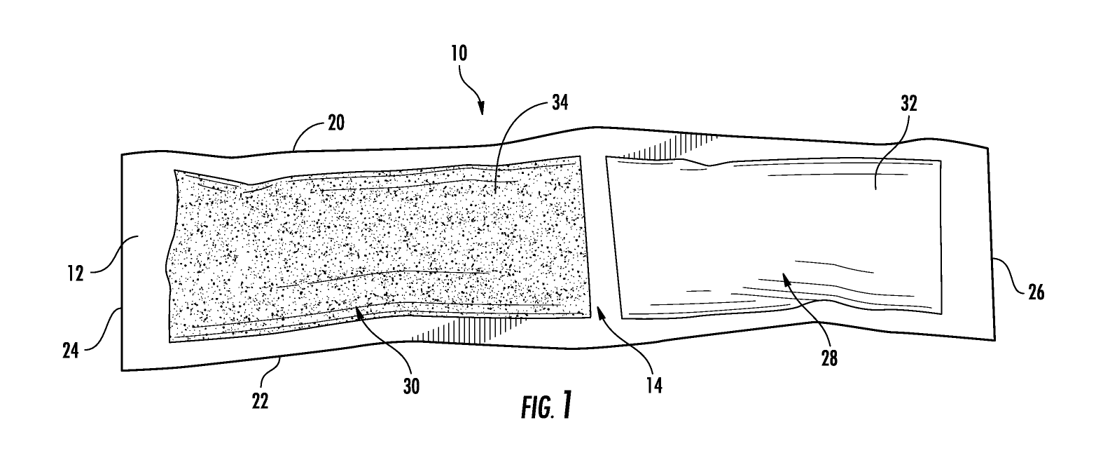

Referring to FIG. 1, a cooling tube 10 includes an outer periphery welded seal 12 and a transverse peelable heat seal 14. In one embodiment cooling tube 10 is formed from two substantially rectangular thermoplastic polymer films 16 and 18. Each polymer film 16 and 18 includes a first pair of parallel sides 20, 22 and a second pair of parallel sides 24, 26 being perpendicular to the first pair of parallel sides 20, 22. The two polymer films 16, 18 are welded about their outer edges 20, 22, 24 and 26 to form a permanent weld between the polymer films. In one embodiment the weld extends inwardly toward the center of each polymer film 16, 18 a distance of 0.5 inches. However other welded widths may be employed as well to provide sufficient weld strength to support a force applied to the cooling tube 10.

Peelable heat seal 14 extends along a vector direction substantially perpendicular to edges 20, 22, and is located between the welded seal portion 20. A first chamber 28 is defined by peelable heat seal 14, and welded seal 12 proximate edge 26 and the welded seal 12 proximate edges 20 and 22 extending from edge 26 to heat seal 14. A second chamber 30 is defined by peelable heat seal 14, and welded seal 12 proximate edge 24 and the welded seal 12 proximate edges 20 and 22 extending from edge 24 to heat seal 14. First chamber 28 contains water 32 and second chamber 30 contains a material 34 which has an endothermic heat reaction of solution in water.

Cooling tube 10 is preferably constructed of a thermoplastic polymer film with a thickness between about 0.004 inches and 0.006 inches; a water vapor transmission rate film at a thickness of 0.005 inches less than about 0.09 g/100 in²/24 hours at 90% Relative Humidity (RH); and a surface which is capable of being heat sealed to itself at less than about 300° F.

In one embodiment the heat seal 14 has a peel strength of less than about 5 lbf/in, more preferably between 1 and 3 lbf/in. It is preferred that this polymer be heat weldable to itself such that a sealed tube created by such welds can support 200 pounds without rupturing or failing. It is also preferred that this film at a thickness of 0.005 inches have an oxygen transmission rate of less than about 0.06 cc/100 in²/24 hours at 0% RH and 23° C. In one embodiment, the polymer film is an adhesive laminate of an abuse resistant higher melting point polymer, typically one with a melt temperature around 500° F. on which aluminum oxide has been vapor deposited to a heat sealable polymer layer with a relatively low heat seal initiation temperature, typically below about 240° F. component with the aluminum oxide being the central layer. A preferred low heat seal initiation temperature component is either ethylene vinyl acetate or linear low density polyethylene (LLDPE) and a preferred abuse resistant higher melting point polymer is a polyester such as polyethylene terephthalate (PET). One embodiment multilayer film is 0.005 inch thick with a central layer of vapor deposited aluminum oxide, one outer layer of PET and the other of a polyolefin containing an LLDPE component.

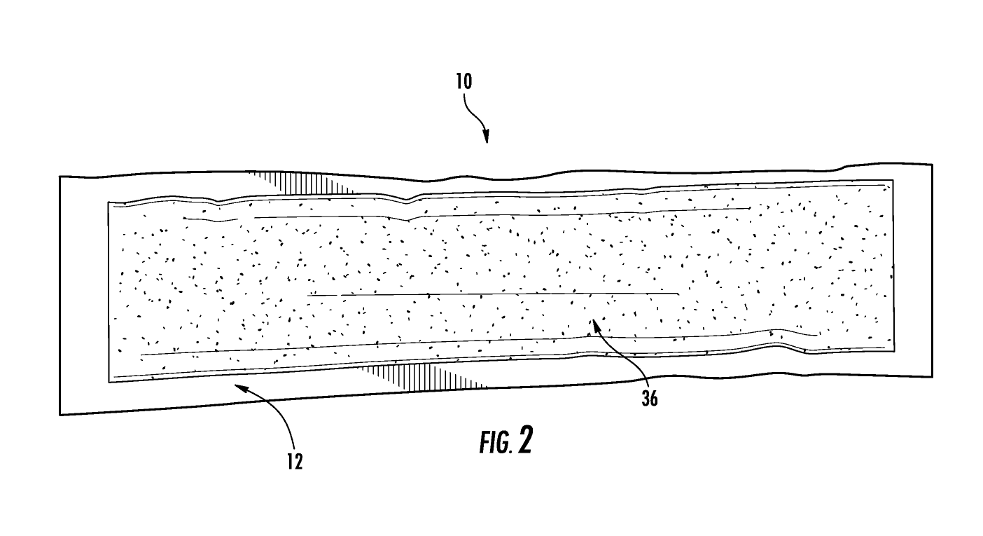

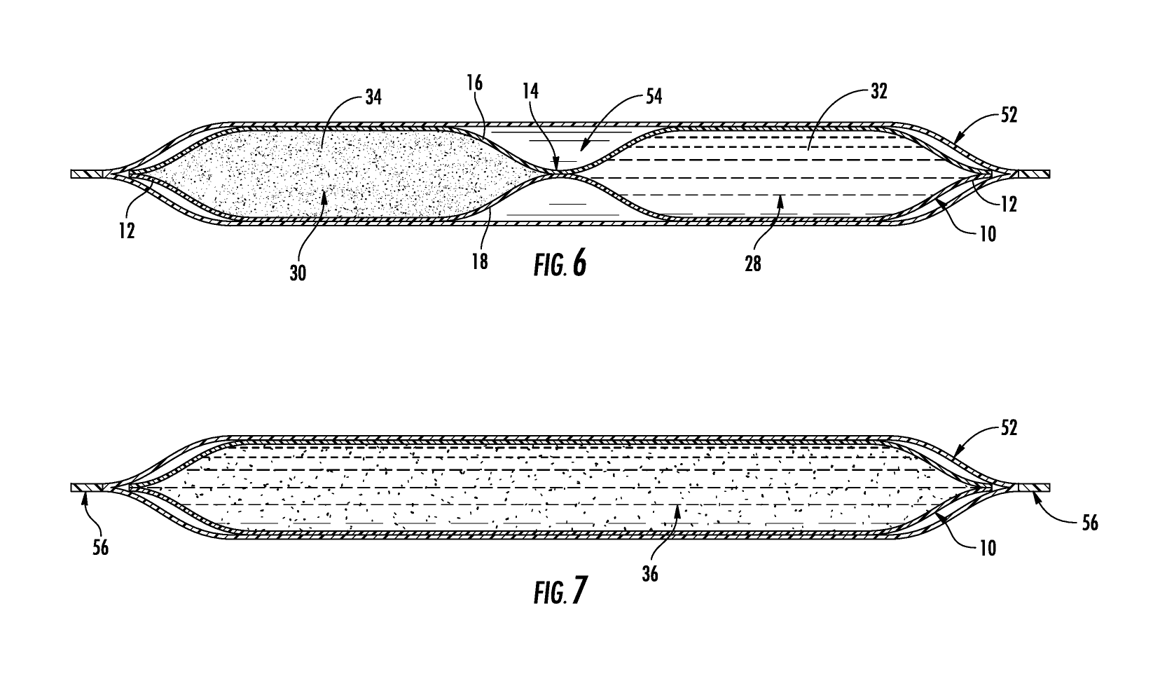

Upon the application of sufficient pressure to the water containing chamber 28 the peelable heat seal 14 is opened resulting in a single chamber 36 with the endotherm generating materials mixed with the water as illustrated in FIGS. 2 and 7. In one embodiment chambers 28, 30 and 36 of cooling tube 10 are defined by a rupture resistant weld 12 such that weld 12 will not fail if it is exposed to a force of at least 200 pounds. Stated another way if a 200 pound weight was placed on cooling tube 10, weld 12 would not fail such that any material or liquid in the cooing tube chambers would not escape.

In one embodiment cooling tubes 10 have two chambers 28, 30 separated by a peelable heat seal 14 with one chamber 28 containing water and the other chamber 30 containing one or more materials 34 which have an endothermic heat of solution in water. Cooling tubes 10 are may be constructed of a polymeric film with good thermal conductivity which has a very low moisture transmission rate and a surface which is readily heat sealed. It is particularly preferred that the moisture transmission rate be low enough to ensure a shelf life of at least about 3 years.

In one embodiment the force needed to open the peelable heat seal 14 is within the manual capability of a normal adult human but also be sufficiently high to avoid accidental activation of a cooling tube during normal handling and transport. The required opening force may be within the manual capability of a frail adult human or an older child to facilitate its use in emergency situations. In one embodiment the force required to open the peelable heat seal is between about 10 and 30 pounds.

In one embodiment each of the cooling tubes 10 preferably contain sufficient materials which have an endothermic heat of solution in water to cause a temperature drop of at least about 30° F. More preferably these materials are non-toxic. In one embodiment the materials are safe and not readily adapted to dangerous uses.

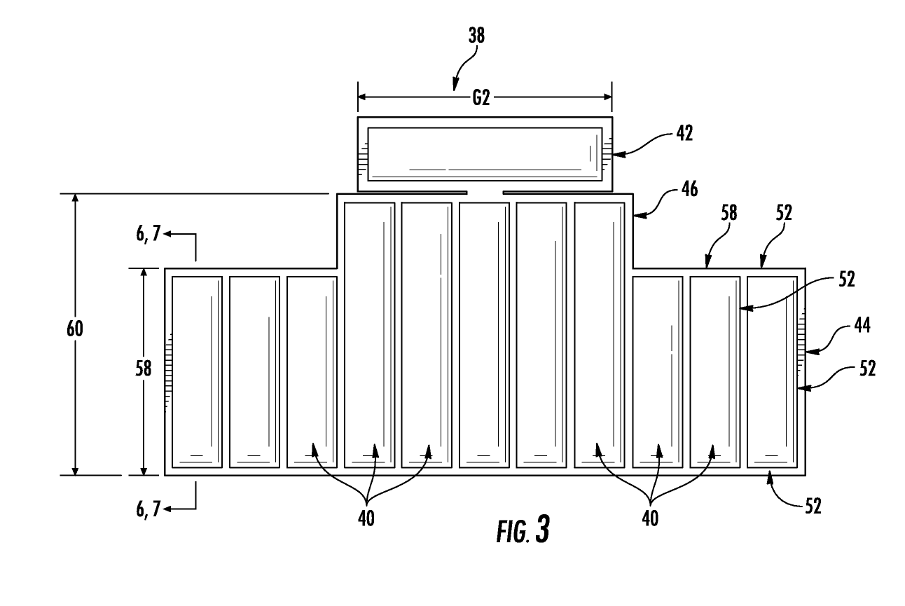

Referring to FIG. 3 a portable self-contained thermal blanket 38 is constructed of a polymer film which has good strength, good thermal conductivity, is heat sealable and is able to withstand a hydrostatic pressure of at least about 200 psi. The polymer film may be a thermoplastic polyurethane, and may be bound to a woven nylon to yield a film with an elongation at break in the warp direction of the nylon weave of around 25% and an elongation at break in the weft direction of the nylon weave of around 46%. In one embodiment this polymer film has a thickness between about 0.009 inches and 0.013 inches. A 0.011 inch thick laminate formed by melt extruding a thermoplastic polyurethane to a woven nylon sheet with 200 denier fibers has been found to be particularly suitable. Blanket 38 is a portable self contained thermal blanket able to provide cooling therapy to a warm blooded animal on its own through the use of materials which have an endothermic heat of solution in water. In one embodiment blanket 38 is sized to the torso of the animal to be treated and has a cooling capacity adequate to significantly affect the core temperature of a patient.

The dimensions of the portable self contained thermal blanket 38 with which the present invention is concerned is adjusted to suit the target of the cooling therapy. A blanket 38 intended for use with an adult human typically is between about 27 inches and 40.5 inches in length and about 48.3 and 72.4 inches in width and contains between about 10 and 14 pockets 40. Pockets 40 are sized to closely fit cooling tubes 10 inserted into them such that adequate force can be applied to the contained cooling tubes 10 to cause their peelable heat seals 14 to peel but also such that the cooling tubes 10 are protected from rupture by external forces. In a preferred embodiment a cooling tube 10 with 28 chamber filled with water will sustain a 20 foot fall to a concrete pad without rupture when sealed into a pocket 40 in thermal blanket 38.

The dimensions of the cooling tubes 10 are adjusted to provide the desired cooling capacity with a reasonable number of such tubes 10 and to provide flexibility in adding additional cooling capacity as needed or desired. For a blanket intended for use with an adult human the individual tubes 10 are typically between about 14 inches and 30 inches in length and about 3.7 inches and 5.6 inches in width. These tubes 10 would each have the capacity to accommodate between about 400 g and 600 g of water in chamber 28 and the appropriate amount of materials with an endothermic heat of solution in water in the chamber 30.

The amount of water and the identity and amount of the materials with an endothermic heat of solution in water are based on the desired cooling characteristics of thermal blanket 38. Cooling therapy requires effective heat transfer from the warm blooded animal being treated to the thermal blanket and this in turn requires that the thermal blanket have an adequate heat capacity. This heat capacity may be provided by cooling an appropriate quantity of water to a low enough temperature. In a preferred embodiment chamber 28 of each cooling tube 10 contains between about 400 g and 600 g of water. The other chamber 30 should then contain a sufficient amount of the materials with an endothermic heat of solution in water to cause a temperature drop of between about to 20° F. and 60° F. It is particularly preferred to use a sufficient amount to cause a temperature drop between about 30° F. and 50° F. In one embodiment the temperature drop from room temperature of approximately 70° F. is 52° F. to about 18° F. Preferred materials are ammonium nitrate and urea. It is particularly preferred to use a combination of urea with KCl and NH₄Cl and it is especially preferred to use acetic acid with the urea, KCl and NH₄Cl. It has been found that the acetic acid accelerates the dissolving of the inorganic salts and ensures their more complete dissolution causing the endothermic reaction to proceed faster and more completely. A mixture of 350 g of urea, 100 g of KCl, 100 g of NH₄Cl and 20 g of acetic acid in one chamber with 500 g of water in the other chamber has been found to be particularly effective.

Chambers 28, 30 of the cooling tubes 10 are established by a heat seal 14 which can be subsequently peeled open by the application of sufficient pressure to chamber 28 containing the water. In a preferred embodiment two walls 16, 18 of a cooling tube 10 which face each other are heat sealed together using a temperature above the heat seal initiation transition temperature of the facing surfaces but below the melting point. The time, temperature and pressure of this sealing operation may be adjusted so that this heat seal can be peeled open by the application of pressure to the water containing chamber 28 which is well within the capacity of an adult human and more preferably a frail adult or older child using his hands but not so low that the seal is liable to open from normal handling transport of the thermal blanket 38. In a preferred embodiment the heat seal 14 is peelable by the application of between about 10 and 30 lbf, more preferably 16 and 24 lbf to the water containing chamber 28 and the seal time between is between about 2 and 5 seconds at a temperature between about 225° F. and 228° F. at a pressure between about 2 and 5 psi. These parameters typically yield a peel strength of about 2 lbf/inch. Precise control of time, temperature and pressure of the sealing operation provides best results.

In a preferred embodiment the outside peripheries of the cooling tubes are sealed by welding together the edges of two equal sized pieces of the thermoplastic film used to form the cooling tubes 10. The outside peripheries extend from edges, 20, 22, 24 and 26 inwardly. The two pieces 16, 18 are placed such that their readily heat sealable surfaces face each other and heat and pressure is applied all along their overlapping peripheries. It is preferred that welds 12 be sufficient to resist a 200 lbf applied to the water containing chamber 28. It has been found particularly convenient that the same polymer film surface be used to create both the peelable heat weld 14 and the highly rupture resistant welds 12 defining the cooling tube 10. For a polymer with a heat sealable layer containing LLDPE a welding seal at a temperature between about 250° F. and 290° F. at a pressure between about 10 and 25 psi has been found to give acceptable welds.

Cooling tubes 10 are placed into appropriately sized pockets 40 in the thermal blanket 38 and the pockets 40 are sealed against any liquid flow from the interior. In a preferred embodiment the pockets 40 are heat sealed. In a particularly preferred embodiment the heat seals are adequate to sustain the blanket being dropped from about 20 feet onto a concrete pad with filled cooling tubes in the pockets with those heat seals. If a cooling tube 10 does rupture and leak, its pocket 40 will maintain the fluid within the pocket and not allow any liquid to escape outside of the pocket 40.

In one embodiment thermal blanket 38 is constructed from a planar material which contains a plurality of pockets 40, each adapted to contain a cooling tube 10, and having good thermal conduction. In one embodiment the planar material is a polymeric material which is strongly resistant to fracture or rupture. Each pocket which contains a cooling tube 10 is sized to provide mechanical support to its cooling tube and is sealed sufficiently to contain any liquid which might leak from its cooling tube. The planar material is a thermoplastic and the pockets are heat sealed. Pockets 40 may be formed by two planar materials being welded together, such as with a heat weld, but other type of welding operations of materials are also contemplated, such as ultrasonic welding. In a particularly preferred embodiment one or more pockets are provided with a temperature sensor. A particularly convenient temperature sensor is a thermochromic ink printed on the exterior of a pocket 40.

Pockets 40 are designed such that sufficient pressure can be exerted on a contained cooling tube 10 to peel open its peelable heat seal 14 while still protecting the cooling tube 10 from rupture should the blanket 38 be subjected to excessive force. Preferably the thermal blanket 38 can be dropped from a height of twenty feet to a concrete pad without any of the cooling tubes contained in the pockets rupturing.

In one embodiment thermal blanket 38 has a sufficient heat capacity to significantly affect the core temperature of the warm blooded animal to whom it is to be applied. This may be achieved by having a sufficient number of cooling tubes 10 with each tube having at least about 400 g of water. It is also contemplated that tubes 10 of different sizes and having differing amounts of water and material be formulated for different pockets 40. In this way the cooling effect may be varied in different parts of the blanket 38.

One embodiment includes a convenient and economical method for the manufacture of portable self contained cooling thermal blankets. It involves partially welding the peripheral edges of two pieces of a thermoplastic film with a very low moisture transmission rate and a readily heat sealable surface to form a cooling tube and dividing the cooling tube into two chambers with a peelable heat seal between a portion of the heat sealable surfaces of these pieces, followed by filling one chamber with water and the other with one or more materials which have an endothermic heat of solution in water and sealing these two chambers by completing the peripheral welds. This is followed by placing cooling tubes 10 in pockets 40 of the planar material described above and sealing the pockets such that it would prevent leakage to the environment of the water of the contained in the cooling tubes should one or more of the cooling tubes rupture. Preferably this planar material is heat sealable and the pockets are heat sealed.

Thermal blankets 38 are preferably made by laminating a thermoplastic polyurethane film to a woven nylon film. In a particularly preferred embodiment two of these laminated films are placed face to face and then welded together in the appropriate places to create a plurality of pockets to accommodate the cooling tubes. It one embodiment the weld creating the pockets and sealing the pockets about the cooling tubes have a peel strength of at least about 44 lbf/inch.

In one embodiment at least some of the pockets 40 are equipped with a temperature sensor. These could include any means of sensing the temperature including a radio frequency (RF) sensor which could be probed for the temperature. In one embodiment a visually readable sensor such as a patch of thermochromic ink which changes color as the temperature changes placed on the exterior of the selected pockets 40 may be used. In another embodiment pockets 40 carrying a temperature sensor are also provided with legible instructions regarding the activation of cooling tubes in response to particular temperature readings.

In one embodiment thermal blankets 38 with cooling tubes 10 sealed in place have a shelf life of at least about 3 years, meaning that the at the end of a three year period after manufacture the materials with an endothermic heat of solution in water still have sufficient activity that the blanket can serve its intended purpose. In one embodiment at the end of this three year period the cooling tubes are still able to display at least a 30° F. drop in temperature when the heat seal 14 between the two chambers 28, 30 is peeled open by the application of an external force.

One embodiment involves partially insulating the cooling tubes 10. It may be advantageous to provide insulation between the pockets 40 containing cooling tubes 10 and the surface of the thermal blanket intended to be distal from the target of the cooling therapy so as to minimize heat transfer from the environment to the cooling tubes. It may also be advantageous to provide insulation between the external portion of pockets 40 intended to be proximate to the target of the cooling therapy so as to avoid exposing the subject to excessively cold temperatures. In either case such insulation may be conveniently provided by placing an insulating foam between a pocket 40 that may be removed as additional cooling of a patient is required. In some cases it may be desirable to provide a removable insulating means between the subject of the cooling therapy and the blanket 38. This may be conveniently provided by pivotally attaching a flap of material with insulating properties to the thermal blanket such that it can be rotated into and out of position between the subject and the surface of the pockets proximate to the subject as desired. This may be helpful when the initial temperature of a cooling tube upon activation is lower than desirable for close contact with the subject but as the cooling tube 10 warms over time such close contact becomes desirable.

Thermal blanket 38 is used by activating one or more cooling tubes 10 and placing it in contact with a warm blooded animal in need of cooling therapy. Cooling tube 10 is activated by applying sufficient force to it within its pocket to cause the opening of the heat seal 14 separating its two chambers 28, 30. Continued use may involve the activation of additional cooling tubes 10 as the need for additional cooling capacity is recognized or the cooling capacity of the initially activated cooling tubes is exhausted. In one embodiment the cooling blanket 38 is placed in contact with the torso of the warm blooded animal undergoing cooling therapy to affect its core temperature. In a further aspect of one embodiment the core temperature of this warm blooded animal is monitored by known medical means such as infrared readings from an eardrum and the activation of cooling tubes is adjusted to obtain a target core temperature. In another aspect of one embodiment a portion of the thermal blanket 38 is put in contact with the head of the subject to minimize injury to the brain.

The cooling therapy provided by the thermal blanket 38 is useful in any emergency or field condition in which slowing the metabolic rate of a warm blooded animal such as a human is indicated to prevent or ameliorate further injury after an initial harmful medical event. The harmful event may arise from the internal functioning of the animal or person, such as a heart attack or stroke, or it may be a traumatic external injury such as a gunshot or other wound or snake bite or it may be exposure to harmful conditions causing reactions such as sunstroke or heat exhaustion.

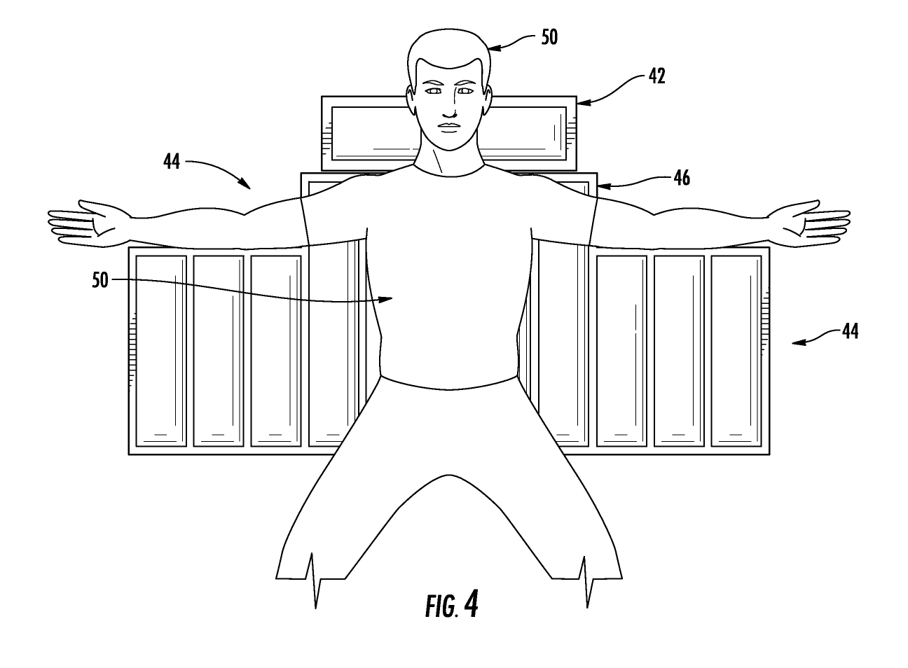

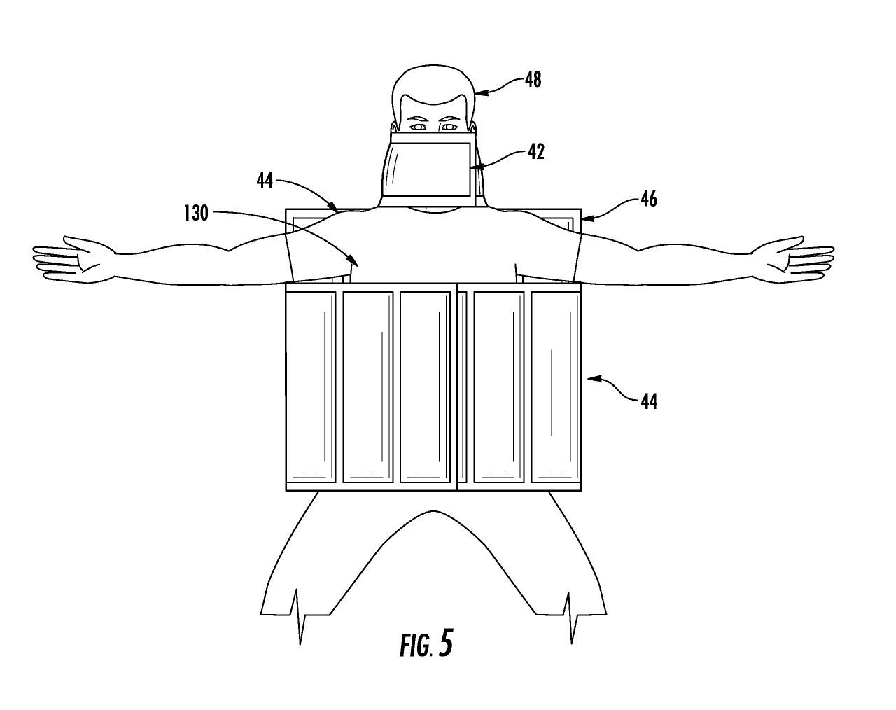

Referring to FIGS. 3-5 a plurality of cooling tubes 10 may be conveniently assembled into a thermal blanket 38 with sections adapted to contact different portions of the body of the animal being subjected to cooling therapy. Thermal blanket 38 has a head wrap section 42, an abdominal wrap section 44 and a central section 46.

The thermal blanket 38 may be applied to an adult human 44 by placing him on the central section 46 such that his head 48 can be wrapped by the a head wrap section 42 and his torso 50 can be wrapped by the abdominal wrap section 44.

Each cooling tube 10 is entirely enclosed by the boundary 52 of its pocket as can be seen in FIGS. 3, 6 and 7 and resides in the interior 54 of this pocket as can be seen in FIG. 6. Each pocket is seal with a rupture resistant weld 56 as illustrated in FIGS. 6 and 7. One convenient design of a thermal blanket 38 for an adult human follows the layout of FIG. 3 wherein the height 58 of abdominal wrap section 44 is about 60 inches and the height 60 of central section 46 is about 33.75 inches. Each cooling tube is placed in a pocket with a margin of 0.75 inches between either an adjacent cooling tube 10 or an exterior edge of the thermal blanket 38. Cooling tubes 10 in both the central section 46 and the abdominal wrap section 44 are about 4.67 inches wide while the central section cooling tubes 10 are 25 inches in height and the abdominal wrap section cooling tubes 18 are 18 inches in height. In one embodiment the cooling tube 10 in head wrap section 42 is 22 inches by 5 inches. It is also contemplated that blanket 38 may have other shapes to fit certain parts of a patient. For example blanket 38 may be configured to wrap a patient's leg/legs or arm/arms The pockets are designed such that sufficient pressure can be exerted on a contained cooling tube to peel open its peelable heat seal while still protecting the cooling tube from rupture should the blanket be subjected to excessive force. Preferably the thermal blanket can be dropped from a height of twenty feet to a concrete pad without any of the cooling tubes contained in the pockets rupturing.

In one embodiment the thermal blanket has a sufficient heat capacity to significantly affect the core temperature of the warm blooded animal to whom it is to be applied. This may be achieved by having a sufficient number of cooling tubes with each tube having at least about 400 g of water.

One embodiment includes a convenient and economical method for the manufacture of portable self contained cooling thermal blankets. It involves partially welding the peripheral edges of two pieces of a thermoplastic film with a very low moisture transmission rate and a readily heat sealable surface to form a cooling tube and dividing the cooling tube into two chambers with a peelable heat seal between a portion of the heat sealable surfaces of these pieces, followed by filling one chamber with water and the other with one or more materials which have an endothermic heat of solution in water and sealing these two chambers by completing the peripheral welds. This is followed by placing cooling tubes in the pockets of the planar material described above and sealing the pockets such that it would prevent leakage to the environment of the water of the contained in the cooling tubes should one or more of the cooling tubes rupture. Preferably this planar material is heat sealable and the pockets are heat sealed.

### EXAMPLES

### Example 1

Cooling Tube. This example illustrates the construction of a cooling tube. A tube was constructed out of a 0.005 inch thick multilayer polymer film made by vapor deposition of an aluminum oxide layer onto a PET film followed by adhesively laminating a polyethylene film with a substantial linear low density polyethylene (LLDPE) content to the PET film such that the aluminum oxide is the central layer to yield a film with a water vapor transmission rate (WVTR) of less than about 0.09 g/100 in²/24 hours at 90% Relative Humidity (RH) and an oxygen transmission rate (OTR) of less than 0.06 cc/100 in²/24 hours at 23° C. and 0% RH. Two rectangular pieces of this multilayer film measuring 4.67 inches by 25 inches were placed one above the other with their edges aligned and the LLDPE containing surfaces facing each other. The long edges were welded together using heat seal bars at about 268° F. applied at a pressure of 16 psi to create a sleeve. Then a heat seal was created across the center of this sleeve perpendicular to the long edges to create two separate chambers by applying a heat seal bar heated to about 226° F. at about 4 psi for about 3 seconds (From previous experience it was known that this heat seal could be opened if one chamber was filled with water, sealed and subjected to about 20 pounds of pressure). Then one chamber was filled with 500 grams of water and sealed by welding the edges of its open end shut with a 268° F. seal bar applied at 16 psi. The other chamber was filled with 350 g of urea, 100 g of KCl, 100 g of NH₄, and 200 g of acetic acid and then sealed in the same manner as the first chamber. The welds along the periphery of the tube were strong enough to resist opening when 180 pounds of force was applied to the water chamber of the cooling tube.

### Example 2

Thermal Blanket. This example illustrates the construction of a thermal blanket. A thermal blanket was constructed of two multilayer films, each made by extrusion laminating a thermoplastic polyurethane (TPU) to a 200 denier woven nylon sheet to yield a 0.011 inch thick composite. The two films were placed one above the other with their edges aligned and their TPU surfaces facing each other. The two composite sheets were the welded to each other to create a plurality of pockets. An appropriately sized cooling tube was placed in each pocket and the pocket was then welded shut. The weld defining each pocket had sufficient strength that when the thermal blanket was dropped from a height of 20 feet onto a concrete pad there was no leakage of the water contained in the cooling tubes sealed in the pockets.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed is:

1. A cooling device comprising:
a first film and a second film welded to the first film forming a tube having an interior;
the tube having a peelable heat seal connecting a portion of the first film with a portion of the second film dividing the interior into a first chamber and a second chamber separated from the first chamber;
the interior of the first chamber including one or more materials which have an endothermic heat of solution in water;
the interior of the second chamber containing water; and
the peelable heat seal being having an adhesive force being overcome by an application of an external force to one of the chambers allowing the material in the first chamber and the water in the second chamber to mix.

2. The cooling device of claim 1 wherein the film is a multilayer flexible thermoplastic polymeric material.

3. The cooling device of claim 2, wherein the multilayer flexible thermoplastic polymeric material includes one layer of polyethylene terephthalate having a layer thereon of vapor deposited aluminum oxide and a layer of polyolefin over the vapor deposited aluminum oxide.

4. The cooling device of claim 3, wherein the material having an endothermic heat of solution in water includes urea, potassium chloride and ammonium chloride.

5. The cooling device of claim 3 wherein the peelable heat seal has an adhesive force of at least 44 lbf/inch

6. The cooling device of claim 5, further including a blanket having a sealed pocket, the container being located within the sealed pocket.

7. A chemically activated thermal blanket comprising:
a. a rupture resistant polymeric film containing a plurality of sealed pockets which contain cooling tubes;
b. the tubes comprising an exterior wall of one or more thermoplastic polymeric films, each having a very low moisture transmission rate and a heat sealable interior surface;
c. the reaction tube being divided into two chambers by a peelable heat seal formed by heat sealing two facing portions of the interior surface or surfaces to each other;
d. one of the chambers containing one or more materials which have an endothermic heat of solution in water and the other of the chambers containing water; and
e. the pockets being sized such that pressure can be applied to the contained cooling tubes to cause the peelable heat seal to open and allow the material or materials and the water to mix but also sized to resist the fracture of the cooling tubes.

8. The thermal blanket of claim 7, having a shelf life of at least three years.

9. The thermal blanket of claim 7, wherein the pockets are sealed such that there is no release to the environment of the contents of any of the cooling tubes.

10. The thermal blanket of claim 7, wherein the cooling tubes can support a weight of 180 pounds.

11. The thermal blanket of claim 7, wherein the rupture resistant polymeric film comprises woven nylon adhered to thermoplastic polyurethane.

12. The thermal blanket of claim 7, wherein the very low moisture transmission rates of the polymeric films forming the exterior walls of the cooling tubes is achieved via a layer of vapor deposited aluminum oxide.

13. The thermal blanket of claim 7, wherein the cooling tubes are formed by welding the peripheral edges of two pieces of the polymeric film together.

14. The thermal blanket of claim 7, wherein the peelable heat seal of the cooling tubes can be readily ruptured with the force applicable by the hands of a normal adult human.

15. The thermal blanket of claim 7, wherein the polymeric films forming the exterior wall of the cooling tubes have a laminate structure in which an aluminum oxide layer is sandwiched between a readily heat sealable layer with a relatively low heat seal initiation temperature component and an abuse resistant layer with a higher melting point or range than the heat sealable layer.

16. The thermal blanket of claim 7 wherein the materials which have an endothermic heat of solution in water comprise urea, potassium chloride and ammonium chloride.

17. The thermal blanket of claim 16 wherein acetic acid has been added to the materials.

18. The thermal blanket of claim 7 wherein a temperature indicator has been applied to the exterior surface of one or more of the sealed pockets.

19. A process for the application of cooling therapy to a warm blooded animal comprising:
a. Providing a thermal blanket which is appropriately sized to the animal to be treated comprising:
i. a rupture resistant polymeric film containing a plurality of sealed pockets which contain cooling tubes;
ii. the tubes comprising an exterior wall of one or more thermoplastic polymeric films, each having a very low moisture transmission rate and a heat sealable interior surface;
iii. each tube being divided into two chambers by a peelable heat seal formed by heat sealing two facing portions of the interior surface or surfaces to each other;
iv. one of the chambers containing one or more materials which have an endothermic heat of solution in water and the other of the chambers containing water; and
v. the pockets being sized such that pressure can be applied to the contained cooling tubes to cause the peelable heat seal to open and allow the material or materials and the water to mix;
b. Applying pressure to at least one of the cooling tubes such that its peelable heat seal opens causing the material or materials and the water to mix without fracturing the cooling tube; and
c. placing the thermal blanket in contact with the warm blooded animal.

20. The process of claim 19 wherein the exterior of one or more of the pockets containing a cooling tube with an opened peelable heat seal carries a temperature sensor and the temperature sensors are used to determine if and when the peelable heat seals of additional cooling tubes in the thermal blanket should be opened as part of the cooling therapy.
